(19) United States
(12) Patent Application Publication
(10) Pub. No.: US 2023/0271028 A1
(43) Pub. Date: Aug. 31, 2023

(54) FAR INFRARED RADIATION DEVICE

(71) Applicant: Brian J. Rice, Las Vegas, NV (US)

(72) Inventor: Brian J. Rice, Las Vegas, NV (US)

(21) Appl. No.: 17/680,814

(22) Filed: Feb. 25, 2022

(51) Int. Cl. A61N 5/06; A61G 13/00

(52) U.S. Cl. CPC A61N 5/0625; A61G 13/009; A61N 2005/066; A61N 2005/0632

(57) ABSTRACT

A portable far infrared (FIR) radiation device containing a conductive heat producing and FIR radiation generating layer containing germanium, conductive carbon powder, a polyurethane resin, a coagulating agent, and a diluent is disclosed. Materials used for making the FIR radiation device are also disclosed.

### FIELD

The present disclosure relates to a portable far infrared (FIR) radiation device providing penetrating heat for therapeutic benefits. The disclosure provides a unique method of generating and delivering FIR radiation to the human body from the device, which can be a yoga mat. Designs and materials used in making the FIR radiation device are also disclosed.

### BACKGROUND

Far infrared (FIR) energy or radiant heat is generally regarded as an electromagnetic energy with a wavelength between 0.75 microns (“µm”) and 1,000 µm. Far infrared light is usually regarded as being found in the range of wavelengths from 5.6 µm to 1000 µm, the longer wavelengths of the infrared spectrum while “near” infrared (IR) and “mid” IR fall in the range of wavelengths shorter than 5.6 µm, and “nearer” the visible light spectrum with respect to frequency and wavelength. The terms “near,” “mid,” and “far” as applied to IR energy refer to their proximity to the visible light spectrum. IR light falls outside the visible spectrum and thus is not visible to the human eye. IR energy is also known as IR radiation, including FIR, and is like the warming rays of the sun. The FIR radiation or the FIR rays, unlike UV radiation, x-rays, or atomic radiation, are safe and often beneficial. When FIR radiation energy penetrates the skin it transfers heat energy, which can penetrate into the body to stimulate and heal tissues, making FIR an effective therapeutic tool for arthritis and tissue injuries among other conditions.

Traditional electric heating mats have considerable power requirements, contain heavyweight gemstones, and are often not portable. Therefore, there is an unmet need for a lightweight rollable and portable FIR radiation generating device at least for the purpose of using as an exercise mat, a yoga mat, or a Pilates mat.

### BRIEF DESCRIPTION OF THE DRAWINGS

Non-limiting and non-exhaustive embodiments of the disclosed FIR radiation device, including the preferred embodiment, are described with reference to the following figures, wherein like reference numerals refer to like parts throughout the various views unless otherwise specified. The embodiments of the invention disclosed herein, and the following detailed description of certain embodiments may be understood with reference to the following drawings:

FIG. 1 illustrates a portable FIR radiation device in the form of a yoga mat.

FIG. 2 depicts different layers of a standard sized (72″ × 24″) FIR radiation yoga mat including the conductive heat producing and FIR radiation generating layer and attached power source inlet.

FIG. 3 illustrates the conductive heat producing and FIR radiation generating layer and attached power source inlet.

FIG. 4 shows a diagram of a power control/timer.

FIG. 5 shows a rear view of a power control/timer.

FIG. 6 shows sketches of top views of a rolled FIR radiation yoga mat.

FIG. 7 illustrates different layers of a smaller sized (24″ × 24″) FIR radiation yoga mat including the conductive heat producing and FIR radiation generating layer and attached power source inlet.

FIG. 8 depicts a side view of the layers of a FIR radiation yoga mat.

FIG. 9 illustrates the power inlet and the energy sources attached to a FIR radiation yoga mat.

### DETAILED DESCRIPTION

This disclosure describes, among other things, a portable far infrared (FIR) radiation device and its use, including various therapeutic applications.

The present application relates to embodiments of a portable FIR radiation device comprising a conductive heat producing and FIR radiation generating layer comprising germanium, conductive carbon powder, a polyurethane resin, a coagulating agent, and a diluent; and two layers of polymeric sheets each comprising independently one or more polymeric materials selected from the group consisting of polyvinyl chloride (PVC), rubber, neoprene, polyurethane, and a synthetic resin. The conductive heat producing and FIR radiation generating layer is sandwiched between the two layers of the polymeric sheets; and the conductive heat producing and FIR radiation generating layer contains an inlet for a power source. The conductive carbon compound converts the electrical energy to thermal energy, thus emitting FIR radiation.

According to one aspect, the disclosed conductive heat producing and FIR radiation generating layer of the FIR radiation device comprises about 14-18 wt% conductive carbon powder, about 0.5-2.5 wt% germanium, about 3-6 wt% coagulating agent, about 15-35 wt% polyurethane resin and about 45-66% diluent selected from the group consisting of ketones, ethanol, toluene, and naphtha.

According to another aspect, the disclosed FIR radiation device weighs about 8-20 lbs.

According to another aspect, the disclosed FIR radiation device is rollable and rolls up to about 8-16 inches in diameter.

One aspect of the disclosure is directed to methods of making a FIR radiation healing device that provides a unique way of generating and delivering FIR radiation to the human body.

Another aspect of the disclosure is directed to the designs and materials used in making the FIR radiation healing device.

Another aspect of the disclosure provides a portable FIR radiation yoga mat that provides penetrating heat for therapeutic benefits, including easing cramping, relaxing mussels, increasing blood flow, and detoxifying a human body in need of such a therapy.

Disclosed herein is a portable far infrared (FIR) radiation device comprising a conductive heat producing and FIR radiation generating layer comprising germanium, conductive carbon powder, a polyurethane resin, a coagulating agent, and a diluent, and two layers of polymeric sheets each comprising independently one or more polymeric materials selected from the group consisting of polyvinyl chloride (PVC), neoprene, polyurethane, thermoplastic elastomer, synthetic resin, rubber, rubber-like material, ethylene vinyl acetate, polymer environmental-friendly resin, or polyester and the like. Preferable polymeric sheet material is polyvinyl chloride (PVC) or rubber, which provides a non-slip surface as well as good elasticity and durability to enable the linear indentations embossed on the surface for repeated folding and unfolding. Commercially available polymeric sheet materials such as closed-cell PVC foam, viscoelastic foam, or other synthetic resins can also be used for these two layers.

In some embodiments, the two layers of polymeric sheets of the FIR radiation device comprise one or more identical polymeric material(s). In other embodiments, the two layers of polymeric sheets of the FIR radiation device comprise one or more different polymeric material(s).

In some embodiments, the conductive heat producing and FIR radiation generating layer is sandwiched between the two layers of the polymeric sheets, with at least one polymeric sheet as the top and one polymeric sheet as the bottom layer.

In another embodiment, the conductive heat producing and FIR radiation generating layer is a few inches shorter than the top and bottom layers. For example, the width of the conductive heat producing and FIR radiation generating layer is about 20″ for a device with a width of 24″ polymeric sheets.

According to one embodiment, 3 M double-sided VHB tape or any compatible tapes or adhesives can be used in between the bottom and top yoga mat layers. Adhesive, for example, can be prepared by mixing 25% to 35% by weight of a polyurethane resin, 20% to 25% by weight of polyester resin, 40% to 50% by weight of ethyl acetate, and the like.

All layers of the FIR radiation device mat are tied with nylon thread looping the top layer with a thickness of, for example, ¼”, and the entire mat with a thickness of, for example, ½”.

In some embodiments, the conductive heat producing and FIR radiation generating layer contains an inlet for a power source and the power supply is crimped through this layer. There are predetermined crimp points at this layer to power the entire device.

In some embodiments, the power source can be an electrical power source connected via an electric cord, a rechargeable battery, or a solar panel.

The power is turned on to produce the conductive heat at the conductive heat producing and FIR radiation generating layer. The carbon compound in this layer converts electrical energy to thermal energy, thereby emitting/generating FIR radiation (infrared rays).

In some embodiments, the conductive heat producing and FIR radiation generating layer comprises about 14-18 wt% conductive carbon powder, about 0.5-2.5 wt% germanium, about 3-6 wt% coagulating agent, about 15-35 wt% polyurethane resin, and about 45-66% diluent selected from the group consisting of ketones, ethanol, toluene and naphtha.

In some embodiments, the conductive heat producing and FIR radiation generating layer comprises about 13-17 wt% carbon powder, about 1-2 wt% germanium, about 10-15 wt% vinyl resin, about 5-10 wt% acrylic resin, about 4-5 wt% coagulating agent, and about 40-60% diluent selected from the group consisting of ethyl acetate, ethanol, toluene, and naphtha.

In another embodiment, the conductive heat producing and FIR radiation generating layer of the device comprises about 0.6-0.7%, 0.7-0.8%, 0.8-0.9%, 0.9-1.0%, 1.0-1.1%, 1.1-1.2%, 1.2-1.3%, 1.3-1.4%, 1.4-1.5%, 1.5-1.6%, 1.6-1.7%, 1.7-1.8%, 1.8-1.9%, 1.9-2.0%, 2.0-2.1%, 2.1-2.2%, 2.2-2.3%, 2.3-2.4%, or 2.4-2.5% by weight of germanium.

In some embodiments, the conductive heat producing and FIR radiation generating layer comprises about 13-17 wt% carbon powder, such as about 13-14 wt%, 14-15 wt%, 15-16 wt%, 16-17 wt%, or 17 wt% carbon powder.

In some embodiments, the conductive heat producing and FIR radiation generating layer comprises about 1-2 wt% germanium, such as about 1.0-1.5 wt% or 1.5-2.0 wt% germanium.

In some embodiments, the conductive heat producing and FIR radiation generating layer comprises about 10-15 wt% vinyl resin, such as about 10 - 11 wt%, 11-12 wt%, 12-13 wt%, 13-14 wt%, or 14-15 wt% vinyl resin.

In some embodiments, the conductive heat producing and FIR radiation generating layer comprises about 5-10 wt% acrylic resin, such as about 5 - 6 wt%, 6-7 wt%, 7-8 wt%, 8-9 wt%, or 9-10 wt% acrylic resin.

In some embodiments, the conductive heat producing and FIR radiation generating layer comprises about 4-5 wt% coagulating agent, such as about 4.0-4.5 wt% or 4.5-5.0 wt% coagulating agent.

In some embodiments, the conductive heat producing and FIR radiation generating layer comprises about 40-60% diluent selected from the group consisting of ethyl acetate, ethanol, toluene, and naphtha.

In some embodiments, the conductive carbon powder in the conductive heat producing and FIR radiation generating layer is in a combination with a polymer resin. Various polymer resins, including polyurethane resin, vinyl resin and acrylic resin can be used in this combination.

In some embodiments, the conductive carbon powder in the conductive heat producing and FIR radiation generating layer is carbon black. Yet in another embodiment, the conductive carbon powder is in combination with ingredients comprising 13-15 wt% carbon black, 10-15 wt% vinyl resin, 5-10 wt% acrylic resin, and a diluent such as ethyl acetate, ethanol, toluene, or naphtha.

According to one embodiment, the conductive heat producing and FIR radiation generating layer has a thickness of about 0.010 to 0.030 inches, about 0.011 to 0.012 inches, about 0.013 to 0.014 inches, about 0.015 to 0.016 inches, about 0.017 to 0.018 inches, about 0.019 to 0.020 inches, about 0.021 to 0.022 inches, about 0.023 to 0.024 inches, about 0.025 to 0.026 inches, about 0.027 to 0.028 inches, or about 0.029 to 0.030 inches. In another embodiment, this layer does not contain an electrical grounding element and is not connected to any electric grounding element.

The top and the bottom layers of the FIR radiation device, for example, the FIR radiation yoga mat, can have a thickness of at least about 0.078” (about 2 mm), at least about 0.118” (about 3 mm), at least about 0.236” (about 6 mm), about 0.250” (about 6.35 mm), or at least about 0.472” (about 12 mm) in a completely unfolded state. The top and the bottom layers of the FIR radiation device in general have a thickness of at most about 0.787” (about 20 mm) in a completely unfolded state.

In some embodiments, the conductive heat producing and FIR radiation generating layer does not contain a gemstone. Unlike conventional heating pads or mats, in this device there are no crystals or aggregates, which is why the device is lightweight, flexible, and portable.

In some embodiments, the FIR radiation device weighs about 8-10 lbs, 10-12 lbs, 12-14 lbs, 14-16 lbs, 16-18 lbs, or 18-20 lbs, depending on the size of the device.

In some embodiments, the FIR radiation device, such as FIR radiating yoga mat in a completely unfolded state, can be of any suitable size, such as a dimension of 72″ length × 24″ width × ½″ thick, dimension of 65″ length × 24″ width × ½″ thick, dimension of 12″ length × 24″ width × ½″ thick, dimension of 10″ length × 20″ width × ½″ thick, dimension of 10″ length × 24″ width × ½″ thick, and of a dimension of any custom size. A standard sized FIR radiating yoga mat with a dimension 72″ length × 24″ width × ½″ thick in a completely unfolded state, according to this invention weighs no more than 16 lbs.

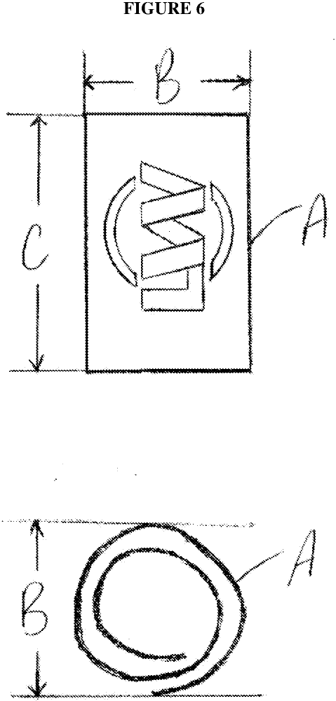

Structurally, in some embodiments, the FIR radiation device is rollable, flexible, bendable, and foldable. As depicted in FIG. 6, the FIR radiation device rolls up to about 8-16 inches in diameter, depending on the size of the device. For example, the device can be rolled to a diameter of up to about 8-10″, 10-12″, 12-14″, or 14-16″.

According to one aspect of the invention, the FIR radiation device is used as an exercise mat, a yoga mat, or a Pilates mat. According to another aspect, the FIR radiation device can be integrated into a massage table or any solid surface.

According to another aspect, the disclosure provides a portable FIR radiation healing yoga mat that provides penetrating heat (for example, from 100° F. to 110° F.) for therapeutic benefits, including easing cramping, easing tight muscles, warming up muscles while stretching, relaxing muscles, increasing blood flow, and detoxifying a human body in need of such a therapy. The FIR radiation heat can penetrate 1, 2, 3, 4, 5, and up to 6 inches into a human body after contact lasting for about 30 minutes to an hour.

According to another aspect, cannabis can be used for enhanced benefits while on a FIR radiation healing device.

The following examples further illustrate the present invention. These examples are intended merely to be illustrative of the present invention and are not to be construed as being limiting.

### EXAMPLES

### 1. Example 1

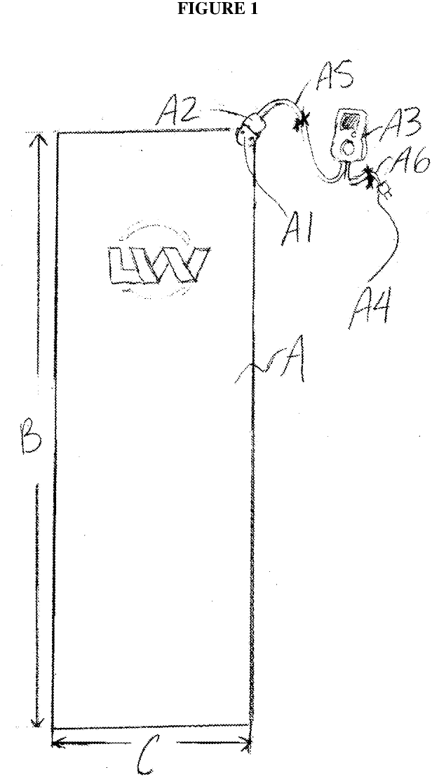

In this Example, FIG. 1 illustrates a portable far infrared (FIR) radiation device in the form of a yoga mat. The figure illustrates a yoga mat (A); a yoga mat power cord mount location with a female side 5-prong removable cord (A1); a detachable 5-prong power cord plug in male side (A2); a control box (A3); a power supply plug, 110V per se (A4); a detachable power cord from the control box to the yoga mat (A5); a detachable power cord from the control box to a wall receptacle cord (A6); a ¼” thick far infrared yoga mat with a length dimension of 72″ (B); and a ¼” thick far infrared yoga mat with a width dimension 24″ (C).

### 2. Example 2

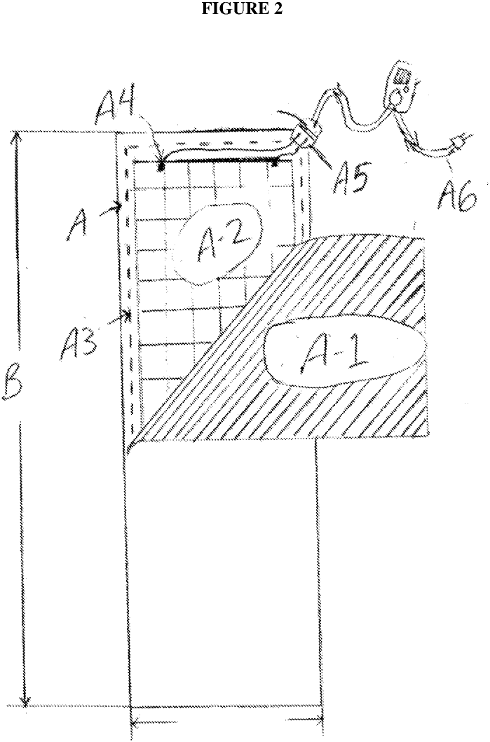

FIG. 2 depicts different layers of a standard sized (72″ × 24″) portable FIR radiation device (in the form of a yoga mat) including the conductive heat producing and FIR radiation generating layer (A2) attached to an inlet for a power source. The depiction of a portable FIR radiation yoga mat prototype shows the top layer of the yoga mat folded over (A1), which is a polymeric sheet comprising one or more polymeric materials such as polyvinyl chloride (PVC), rubber, neoprene, polyurethane, and/or a synthetic resin. Marked as A2 is a flooring section (about 20″ × 60″, 13 watts per sq. ft. 12 sq. ft. 156 watts, 1.26 amps), which is a conductive heat producing and FIR radiation generating layer (A2) comprising germanium, conductive carbon powder, polyurethane resin, a coagulating agent, and a diluent. The bottom layer (A) is also a polymeric sheet comprising one or more polymeric materials such as polyvinyl chloride (PVC), rubber, neoprene, polyurethane, and/or a synthetic resin. A2 layer is sandwiched between the two layers of the polymeric sheets, marked as A and A1. Marked as A3 is an adhesive (such as 3 M double-sided VHB tape) binding the top (A1) and the bottom (A) layers. FIG. 2 also depicts a power supply split, 110V and crimped to the mat (A4); a detachable power cord extending from the control box to a wall receptacle (A5); a detachable power cord extending from the control box to a wall receptacle (A6); yoga mat length dimension 72″ (B); and the width dimension 24″ (C). 3 M double-sided VHB tape can be replaced with any compatible types of tapes or adhesives. Adhesive, for example, can be prepared by mixing 25% to 35% by weight of a polyurethane resin, 20% to 25% by weight of a polyester resin, 40% to 50% by weight of ethyl acetate, and the like.

### 3. Example 3

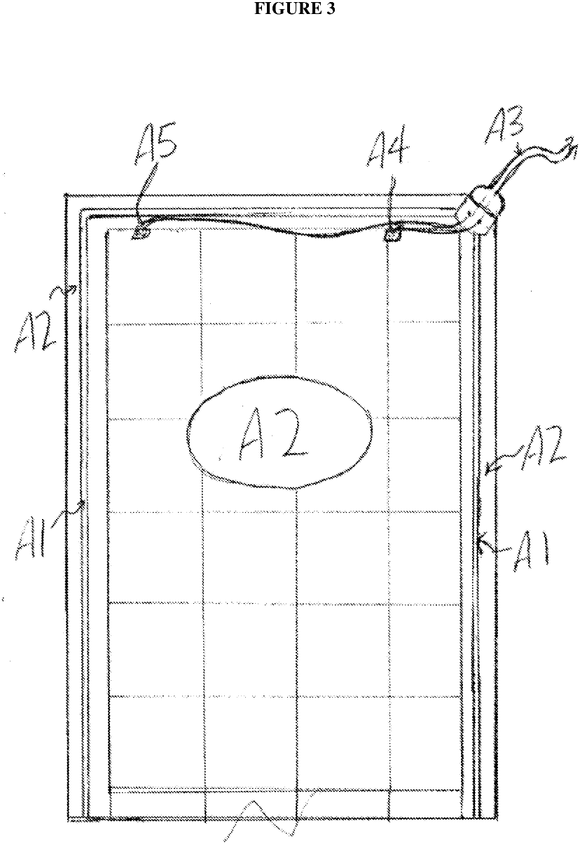

FIG. 3 illustrates the conductive heat producing and FIR radiation generating layer and attached power source inlet. The depiction of a ¼” yoga mat prototype shows the conductive heat producing and FIR radiation generating layer attached (A2) to the bottom layer polymeric sheet with a 3 M double-sided VHB tape (A1); a flooring section of the radiation generating layer (about 20″ × 60″, 13 watts per sq. ft. 12 sq. ft. 156 watts, 1.26 amps) (A2); a power cord to control box (A3); a 110 V power supply split and crimped to the mat (A4). The FIR radiation mat has predetermined crimp points to power the entire mat. 3 M double-sided VHB tape can be replaced with any compatible types of tapes or adhesives. Adhesive, for example, can be prepared by mixing 25% to 35% by weight of a polyurethane resin, 20% to 25% by weight of a polyester resin, 40% to 50% by weight of ethyl acetate, and the like.

### 4. Example 4

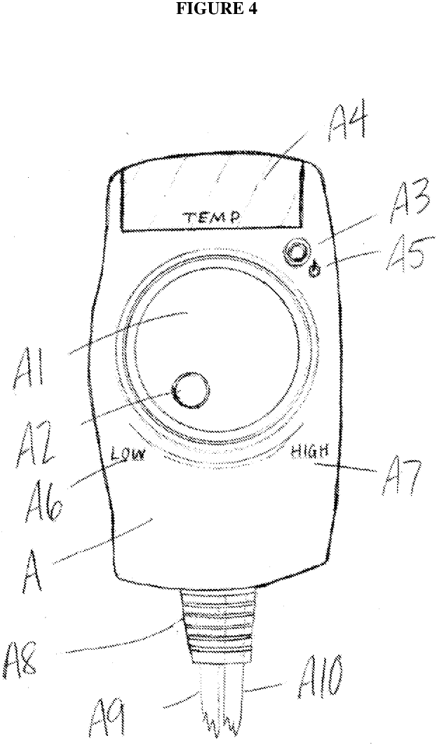

FIG. 4 is a diagram of a power control/timer showing a rotating temperature selector (A1), a finger depression to control temperature (A2), a power on or off button (A3), a digital temperature LED number reading window (A4), a power button indicator (A5), a low temperature setting indicator (A6), a high temperature setting indicator (A7), a power cord attachment to control box (A8), a power cord attachment to wall receptacle, 110V or 240V (A9), and a power cord attachment to yoga mat detachable plug (A10).

### 5. Example 5

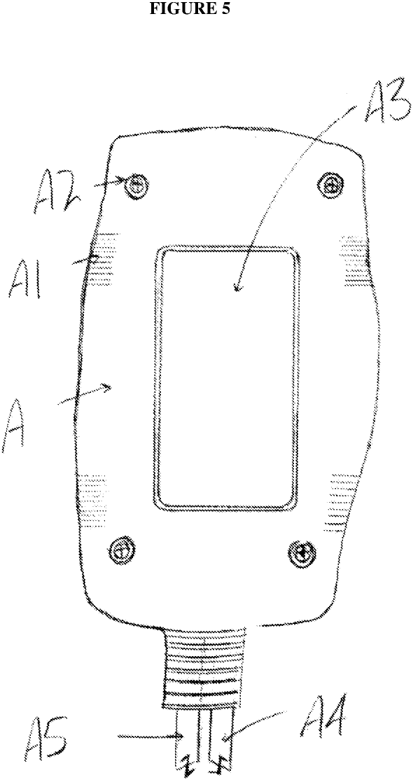

FIG. 5 is a rear view of a power control/timer showing vents in the timer body (A1), ¾” Phillips head fasteners (A2), an embossed depression for a sticker (A3), a power cord attachment to a wall receptacle, 110 V or 240 V (A4), and a power cord attachment to a yoga mat detachable plug (A5).

### 6. Example 6

FIG. 6 shows sketches of top views of a rolled FIR radiation yoga mat (A), depicting the width of the rolled FIR radiation yoga mat (15″) (B) and the standing height (24″) (C) of the rolled yoga mat.

### 7. Example 7

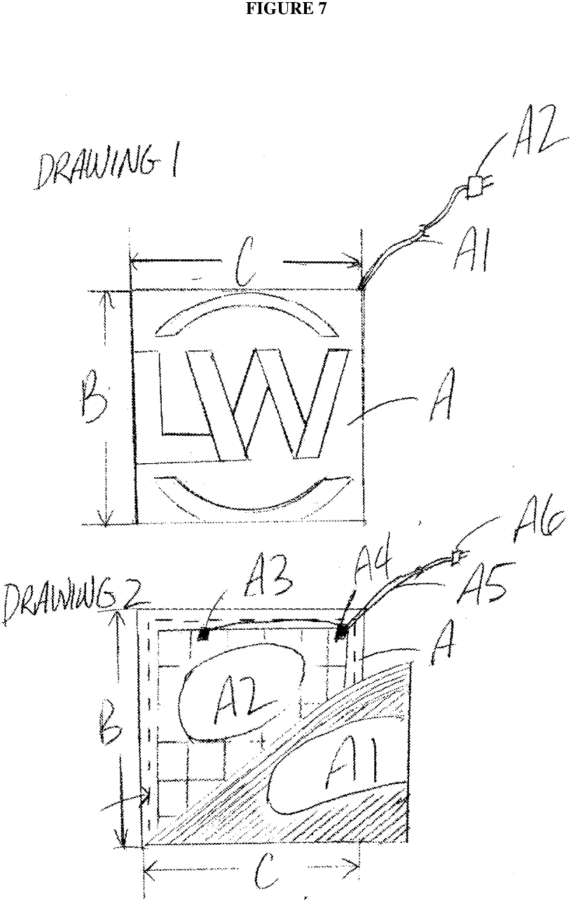

FIG. 7 illustrates different layers of a smaller sized (24″ × 24″) FIR radiation yoga mat including an exposed conductive heat producing and FIR radiation generating layer and an attached power source inlet.

Drawing 1: a smaller sized (24″ × 24″) FIR radiation yoga mat (A) with a power cord (A1), where the width of the yoga mat is 24″ (B) and the length of the yoga mat is 24″ (C).

Drawing 2: a smaller sized (24″ × 24″) FIR radiation yoga mat (A), depicting the inside of the top layer of the yoga mat (A1), a heated FIR element (A2), a crimp point to power element (A3 and A4), a power cord (A5), and a wall plug, no ground (A6), where the width of the yoga mat is 24″ (B) and the length of the yoga mat is 24″ (C).

### 8. Example 8

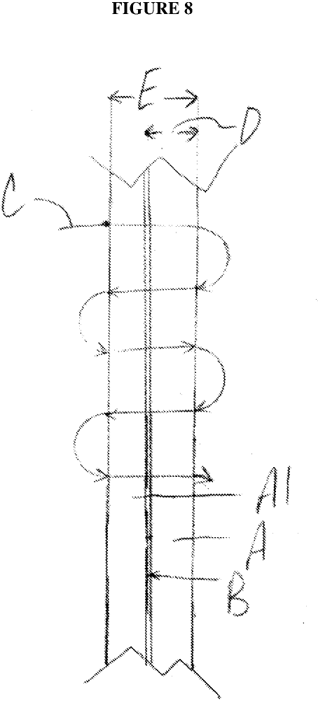

FIG. 8 depicts a side view of the layers of a FIR radiation yoga mat: a side view of the bottom layer (A), a side view of the top layer (A1), 3 M double-sided VHB tape (B) in between the bottom and top yoga mat layers, all layers of the FIR radiation yoga mat are tied with nylon thread looping (C), the top layer with a thickness of ¼” (D), and the entire mat with a thickness of ½” (E). 3 M double-sided VHB tape can be replaced with any compatible tapes or adhesives. Adhesive, for example, can be prepared by mixing 25% to 35% by weight of a polyurethane resin, 20% to 25% by weight of a polyester resin, 40% to 50% by weight of ethyl acetate, and the like.

### 9. Example 9

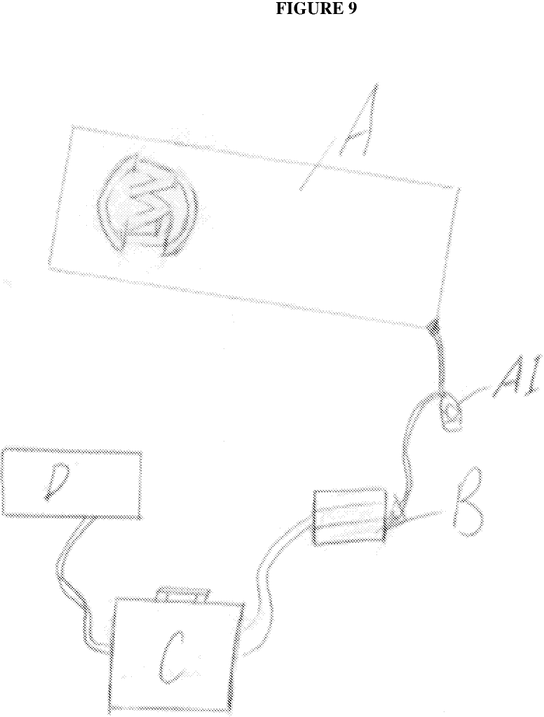

FIG. 9 illustrates the power inlet and various energy sources attached to a FIR radiation yoga mat of about 12.0 lbs. in weight (A), which includes a power controller (A1), a 1500 watt power converter of about 3.0 lbs. in weight (B), a Jackery Solar Generator 1000 (Jackery 1000 W and 2 packs SolarSaga 100 W), a Rebelcell outdoor box 12.7V charger of about 14.0 lbs. in weight or a 12.6V20A NMC charger of about 1.0 lbs. in weight (C), and a solar panel to recharge the outdoor box (D) [Jackery Solar Sega 100-watt solar panels, 4 each, 10 lbs. each].

The yoga mat is portable and uses only 13 watts of power per square feet. This allows it to be used in places without shore power. The battery and charger with converters will power the yoga mat for approximately four hours, depending on outside temperatures and the ground temperature. The solar panels will charge the battery until dark, and then there are four hours of power still available.

What is claimed is:

1. A portable far infrared (FIR) radiation device, wherein the device comprises:
a conductive heat producing and FIR radiation generating layer comprising germanium, conductive carbon powder, polyurethane resin, a coagulating agent, and a diluent,
two layers of polymeric sheets each independently comprising one or more polymeric materials selected from the group consisting of polyvinyl chloride (PVC), rubber, neoprene, polyurethane, and a synthetic resin,
wherein the conductive heat producing and FIR radiation generating layer is sandwiched between the two layers of the polymeric sheets; and
wherein the conductive heat producing and FIR radiation generating layer contains an inlet for a power source.

2. The FIR radiation device of claim 1, wherein the conductive heat producing and FIR radiation generating layer comprises about 14-18 wt% conductive carbon powder, about 0.5-2.5 wt% germanium, about 3-6 wt% coagulating agent, about 15-35 wt% polyurethane resin and about 45-66% diluent selected from the group consisting of ketones, ethanol, toluene and naphtha.

3. The FIR radiation device of claim 1, wherein the conductive heat producing and FIR radiation generating layer comprises about 13-17 wt% carbon powder, about 1-2 wt% germanium, about 10-15 wt% vinyl resin, about 5-10 wt% acrylic resin, about 4-5 wt% coagulating agent, and about 40-60% diluent selected from the group consisting of ethyl acetate, ethanol, toluene, and naphtha.

4. The FIR radiation device of claim 1, wherein the two layers of polymeric sheets comprise one or more identical polymeric material(s).

5. The FIR radiation device of claim 1, wherein the two layers of polymeric sheets comprise one or more different polymeric material(s).

6. The FIR radiation device of claim 1, wherein the device is an exercise mat, a yoga mat, or a Pilates mat.

7. The FIR radiation device of claim 1, wherein the conductive carbon powder is in combination with a polymer resin.

8. The FIR radiation device of claim 7, wherein the polymer resin is selected from the group consisting of polyurethane resin, vinyl resin, and acrylic resin.

9. The FIR radiation device of claim 1, wherein the conductive carbon powder is carbon black.

10. The FIR radiation device of claim 1, wherein the conductive carbon powder is in combination with ingredients comprising 13-15 wt% carbon black, 10-15 wt% vinyl resin, 5-10 wt% acrylic resin, and a diluent selected from the group consisting of ethyl acetate, ethanol, toluene and naphtha.

11. The FIR radiation device of claim 1, which weighs about 12-16 lbs.

12. The FIR radiation device of claim 1, which is rollable and rolls up to about 12-15 inches in diameter.

13. The FIR radiation device of claim 1, wherein the conductive heat producing and FIR radiation generating layer does not contain an electrical grounding element.

14. The FIR radiation device of claim 1, wherein the FIR radiation generating layer does not contain a gemstone.

15. The FIR radiation device of claim 1, wherein the device is integrated into a massage table.
